(19) United States
(12) Patent Application Publication
(10) Pub. No.: US 2025/0376521 A1
(43) Pub. Date: Dec. 11, 2025

(54) Siglec Receptor Checkpoint Inhibitors and Method of Using Them to Inhibit Neoplastic Cell Growth

(71) Applicant: Advantigen Biosciences, LLC, Madison, WI (US)

(72) Inventors: Mark Jackson, Madison, WI (US); Heather Kirkpatrick, Madison, WI (US); Fred Hyde, Madison, WI (US)

(73) Assignee: Advantigen Biosciences, LLC, Madison, WI (US)

(21) Appl. No.: 18/693,819

(22) PCT Filed: Sep. 29, 2022
(86) PCT No.: PCT/US2022/077257

§ 371 Date: Mar. 20, 2024

Related U.S. Application Data
(60) Provisional application No. 63/250,630, filed on Sep. 30, 2021.

(51) Int. Cl. C07K 16/28; A61P 35/00; A61P 37/04; G01N 33/68

(52) U.S. Cl. CPC C07K 16/2803; A61P 35/00; A61P 37/04; C07K 16/2818; G01N 33/6893; C07K 2317/14; C07K 2317/24; C07K 2317/76; C07K 2317/92; G01N 2333/70503; G01N 2800/52

(57) ABSTRACT

The present disclosure provides compositions for inhibiting interaction of Siglec-5 with its cognate ligand on cancer cells and their use as checkpoint inhibitors in the treatment of neoplastic disorders. A method for the treatment and/or prevention of a cancer that expresses a Siglec-5 ligand in a subject is provided comprising administering to the subject a molecule that inhibits the interaction between Siglec-5 and a Siglec-5 ligand expressed on the cancer cell. In some aspects, the subject is identified as having a cancer expressing a Siglec-5 ligand. Preferably, the molecule is a monoclonal antibody against Siglec-5.

### CROSS-REFERENCE TO RELATED APPLICATIONS

This application claims the benefit of U.S. Provisional Patent Application No. 63/250,630, filed Sep. 30, 2021, the full disclosure of which is incorporated herein by reference.

### BACKGROUND

Improving treatment of disease has been a long running theme in both basic and clinical research. Therapies for specific disease targets as well as targets to modulate the immune system have been the goals of researchers and such research for a very long time. Immune checkpoints are pathways with inhibitory or stimulatory features that maintain self-tolerance and assist with immune response. They include the ability to immunomodulate cells, groups of cells, tissues, groups of tissues in an in vitro manner, and in vivo in an animal or the immune system of an animal. New targets for immune modulation have been mis-categorized in the past, but have now been characterized as immune checkpoint cell surface receptors which can be targeted by checkpoint inhibitors. Targeted treatment can affect cancer and increase the effectiveness of CAR-T therapies for solid tumor cancers,

There is an unmet need in the art for checkpoint inhibitors such as a monoclonal antibody to modulate the immune system.

### SUMMARY OF THE INVENTION

Siglec-5 is identified herein as an immune checkpoint inhibitor. Provided herein are methods for modulating an immune response in a subject comprising administering to the subject a molecule that inhibits the interaction between Siglec-5 and a cognate Siglec-5 ligand (e.g., a Siglec-5 ligand expressed on a cancer cell) such that the immune response in the subject is modulated. In some aspects, the Siglec-5 ligand is a (glyco)protein ligand. Preferably, the molecule enhances, stimulates or increases the immune response in the subject.

In some aspects, a cancer cell that expresses one or more Siglec-5 ligands is identified by the methods described in Vuchkovska et al., Immunology, 166(2):238-248 (2022), the entire contents of which are incorporated herein by reference. In related aspects, inhibition of an interaction between Siglec-5 and a Siglec-5 ligand expressed on a cancer cell by a molecule (e.g., an anti-Siglec-5 antibody) is assessed by the methods described in Vuchkovska et al. In other aspects, stimulation of an anti-tumor T cell response by a molecule that inhibits interaction between Siglec-5 and a Siglec-5 ligand expressed on a cancer cell is assessed by the methods described in Vuchkovska et al.

In some aspects, a method for the treatment and/or prevention of a cancer that expresses a Siglec-5 ligand in a subject is provided comprising administering to the subject a molecule that inhibits the interaction between Siglec-5 and a Siglec-5 ligand expressed on the cancer cell. In some aspects, the subject is identified as having a cancer expressing a Siglec-5 ligand.

In certain embodiments, the cancer treated with the molecule (e.g. anti-Siglec-5 antibody or soluble form of Siglec-5) that inhibits the interaction between Siglec-5 and a Siglec-5 ligand expressed on the cancer cell molecule, includes but is not limited to, a solid tumor, a hematological cancer (e.g., leukemia, lymphoma, myeloma, e.g., multiple myeloma), and a metastatic lesion. In one embodiment, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal-cell carcinoma, liver cancer, non-small cell lung cancer, cancer of the small intestine and cancer of the esophagus. The cancer may be at an early, intermediate, late stage or metastatic cancer.

In one embodiment, the cancer is chosen from a lung cancer (e.g., lung adenocarcinoma or a non-small cell lung cancer (NSCLC) (e.g., a NSCLC with squamous and/or non-squamous histology, or a NSCLC adenocarcinoma)), a melanoma (e.g., an advanced melanoma), a renal cancer (e.g., a renal cell carcinoma), a liver cancer (e.g., hepatocellular carcinoma), a myeloma (e.g., a multiple myeloma), a prostate cancer, a breast cancer (e.g., a breast cancer that does not express one, two or all of estrogen receptor, progesterone receptor, or Her2/neu, e.g., a triple negative breast cancer), an ovarian cancer, a colorectal cancer, a pancreatic cancer, a head and neck cancer (e.g., head and neck squamous cell carcinoma (HNSCC), anal cancer, gastro-esophageal cancer (e.g., esophageal squamous cell carcinoma), mesothelioma, nasopharyngeal cancer, thyroid cancer, cervical cancer, a lymphoproliferative disease (e.g., a post-transplant lymphoproliferative disease) or a hematological cancer, (e.g., diffuse large B cell lymphoma, T-cell lymphoma, B-cell lymphoma, or a non-Hodgkin lymphoma), or a leukemia (e.g., a myeloid leukemia or a lymphoid leukemia).

In some preferred embodiments, the cancer is selected from colorectal cancer, esophageal cancer, squamous cell carcinoma, hepatocellular carcinoma, Hodgkin lymphoma, head and neck squamous cell carcinoma, melanoma, mesothelioma, non-small cell lung cancer, renal cell carcinoma, urothelial carcinoma, breast cancer, cervical cancer, cutaneous squamous cell carcinoma, endometrial carcinoma, esophageal carcinoma, gastric carcinoma, Merkel cell carcinoma, large B cell lymphoma, and small cell lung cancer. In some aspects, a method for treatment of one or more of these cancers comprises a step of co-administering to a subject in need thereof (i) a molecule that inhibits binding between Siglec-5 and a Siglec-5 ligand expressed on a cancer cell and (ii) one or more additional immune checkpoint inhibitors, preferably comprising an inhibitor of PD-1.

In some embodiments, the subject comprises a cancer microenvironment having an elevated level of PD-L1 expression. Alternatively, or in combination, the cancer microenvironment can have increased IFNγ. and/or CD8 expression.

In some embodiments, the subject has, or is identified as having, a tumor that has one or more of high PD-L1 level or expression, or as being Tumor Infiltrating Lymphocyte (TIL)+ (e.g., as having an increased number of TILs), or both. In certain embodiments, the subject has, or is identified as having, a tumor that has high PD-L1 level or expression and that is TIL+. In some embodiments, the methods described herein further include identifying a subject based on having a tumor that has one or more of high PD-L1 level or expression or as being TIL+, or both. In certain embodiments, the methods described herein further include identifying a subject based on having a tumor that has high PD-L1 level or expression and as being TIL+. In some embodiments, tumors that are TIL+ are positive for CD8 and IFNγ. In some embodiments, the subject has, or is identified as having, a high percentage of cells that are positive for one, two or more of PD-L1, CDS, and/or IFNγ. In certain embodiments, the subject has or is identified as having a high percentage of cells that are positive for all of PD-L1, CD8, and IFNγ.

In some embodiments, the methods described herein further include identifying a subject based on having a high percentage of cells that are positive for one, two or more of PD-L1, CD8, and/or IFNγ. In certain embodiments, the methods described herein further include identifying a subject based on having a high percentage of cells that are positive for all of PD-L1, CD8, and IFNγ. In some embodiments, the subject has, or is identified as having, one, two or more of PD-L1, CD8, and/or IFNγ, and one or more of a lung cancer, e.g., squamous cell lung cancer or lung adenocarcinoma; a head and neck cancer; a squamous cell cervical cancer; a stomach cancer; an esophageal cancer; a thyroid cancer; a melanoma, and/or a nasopharyngeal cancer (NPC). In certain embodiments, the methods described herein further describe identifying a subject based on having one, two or more of PD-L1, CD8, and/or IFNγ, and one or more of a lung cancer, e.g., squamous cell lung cancer or lung adenocarcinoma; a head and neck cancer; a squamous cell cervical cancer; a stomach cancer; a thyroid cancer; a melanoma, and/or a nasopharyngeal cancer.

In some embodiments, a subject has, or is identified as having, a tumor that has one, two, or more of high PD-1 level or expression, high TIM-3 level or expression, and/or high level of infiltration of regulatory T cells in the tumor, e.g., an increased number or percentage of Tregs present in the tumor. In certain embodiments, the subject has, or is identified as having, a tumor that has a high level or expression of PD-1 and TIM-3, and a high level, e.g., number, or regulatory T cells in the tumor. In some embodiments, the methods described herein further include identifying a subject based on one, two or more of a high percentage of cells that are positive for PD-1, a high percentage of cells that are positive for TIM-3, and/or a high level of infiltration of regulatory T cells in the tumor, e.g., an increased number or percentage of Tregs present in the tumor. In some embodiments, the methods described herein further include identifying a subject based on one, two or more of a high percentage of cells that are positive for PD-1, a high percentage of cells that are positive for TIM-3, and/or a high level of infiltration of regulatory T cells in the tumor, e.g., an increased number or percentage of Tregs present in the tumor, and one or more of a lung cancer, e.g., non-small cell lung cancer (NSCLC); a hepatocellular cancer, e.g., hepatocellular carcinoma; or an ovarian cancer, e.g., ovarian carcinoma.

In some embodiments, the subject to be treated according to the present methods is identified as having a tumor microenvironment comprising an elevated level of PD-L1 and/or PD-1 expression.

In some embodiments, the subject to be treated according to the present methods is identified as having a tumor microenvironment comprising an elevated level of expression of a Siglec-5 ligand. In related embodiments, the subject to be treated according to the present methods is identified as having a tumor microenvironment comprising an elevated level of expression of a Siglec-5 ligand and PD-1.

Also provided is a method of treating a cancer, comprising: testing a sample (e.g., a sample from a human subject comprising cancer cells) for the presence of a Siglec-5 ligand and/or PD-1, thereby identifying a Siglec-5 ligand and/or PD-1 value, comparing the Siglec-5 ligand and/or PD-1 value to a control value, and if the Siglec-5 ligand and/or PD-1 value is greater than the control value, administering a therapeutically effective amount of an anti-Siglec-5 antibody (e.g., an anti-Siglec-5 antibody described herein) to the subject, optionally in combination with one or more other agents, e.g., an anti-PD-1 antibody molecule, thereby treating the cancer.

In some aspects, hypersialylation of a cancer cell modulates the interaction between the cancer cell and Siglec-5.

In some aspects, a molecule that inhibits the interaction between Siglec-5 and a Siglec-5 ligand expressed on a cancer cell comprises a soluble form of Siglec-5 which may act as a decoy receptor for Siglec-5 ligand. In some embodiments, soluble Siglec-5 comprises a Siglec-5 isoform encoding a soluble truncated protein having the extracellular sequence of Siglec-5, e.g., as described in Connolly et al., Br J Haematol., 119(1):221-238 (2002). In other embodiments, soluble Siglec-5 comprises a portion of the extracellular sequence of Siglec-5 comprising a ligand-binding domain. In a related aspect, soluble Siglec-5 comprises the amino terminal V-set immunoglobulin domain that recognizes sialic acids. In some embodiments, the soluble form of Siglec-5 comprises a fusion protein comprising (i) the extracellular portion of Siglec-5 or a ligand-binding fragment thereof and (ii) an Fe portion of IgG1.

In other aspects, a molecule that inhibits the interaction between Siglec-5 and a Siglec-5 ligand expressed on a cancer cell comprises an antibody or antigen-binding fragment thereof. In some embodiments, the antibody binds specifically to Sigle-5 and reduces binding of Siglec-5 to a Siglec-5 ligand. In preferred embodiments, the antibody or antigen binding fragment thereof is administered to a subject for use in the treatment and/or prevention of cancer in the subject. In some preferred embodiments, the antibody or antigen-binding fragment binds to Siglec-5 and reduces binding of Siglec-5 to a Siglec-5 ligand. In other embodiments, the antibody or antigen-binding fragment thereof binds to a Siglec-5 ligand expressed on a cancer cell and reduces binding of Siglec-5 to the Siglec-5 ligand.

In other aspects, a molecule that inhibits the interaction between Siglec-5 and a Siglec-5 ligand expressed on a cancer cell comprises an interfering RNA or antisense RNA. In some aspects, the interfering RNA or antisense RNA reduces expression of Siglec-5 in a tumor microenvironment.

In other aspects, a molecule that inhibits the interaction between Siglec-5 and a Siglec-5 ligand expressed on a cancer cell comprises a small molecule. See, e.g., Rillahan et al., Angew Chem Int Ed Engl., 51(44):11014-11018 (2012), the contents of which are incorporated herein by reference.

Also provided is a combination therapy comprising the co-administration of (i) a molecule that inhibits binding of Siglec-5 to a Siglec-5 ligand and (ii) a second cancer therapeutic. In some embodiments, a molecule that inhibits binding of Siglec-5 is administered to a subject in combination with a modulator of a costimulatory molecule (e.g. an agonist of a costimulatory molecule). In other embodiments, a molecule that inhibits binding of Siglec-5 is administered to a subject in combination with a modulator of an inhibitory molecule (e.g. an inhibitor of an immune checkpoint protein). In some aspects, the second cancer therapeutic is an immune checkpoint inhibitor, preferably an anti-PD-1, anti-PD-L1 and/or anti-CTLA-4 antibody.

Also provided herein are isolated antibodies that bind to Siglec-5. In some embodiments, the isolated antibodies are human monoclonal antibodies that bind to human Siglec-5. Also provided are nucleic acid molecules encoding the antibodies, and pharmaceutical compositions comprising the antibodies. In some embodiments, an anti-Siglec-5 antibody provided herein exhibits one or more of the following properties:
(a) Binds to human Siglec-5 with a kD of [1×10−7 M] or less
(b) Does not substantially reduce cell surface levels of Siglec-5 expressed on an immune cell (e.g. activated T cell) in vitro and/or in vivo
(c) Increases T-cell proliferation in a Mixed Lymphocyte Reaction (MLR) assay
(d) Increases IL-2 secretion in a MLR assay
(e) Increases T-cell proliferation and/or IL-2 secretion in a T lymphocyte proliferation assay
(f) Binds to human Siglec-5 and cynomolgus monkey Siglec-5
(g) Inhibits binding of Siglec-5 to a Siglec-5 ligand expressed on an immune cell
(h) Inhibits tumor cell growth in vivo
(i) Does not cross-react with human Siglec-14

Preferably, the anti-Siglec-5 antibody is a human antibody, although in alternative embodiments, the antibody can be, for example, a murine antibody, a chimeric antibody, or a humanized antibody. Antibodies of the invention can be, for example, full-length antibodies, for example of an IgG1 or IgG4 isotype. Alternatively, the antibodies can be antibody fragments such as an Fab fragment (monovalent fragment consisting of the VL, VH, CL and CH1 domains), an F(ab′)2 fragment (bivalent fragment comprising two Fab fragments linked by at least one disulfide bridge at the hinge region), a Fd fragment (consisting of the VH and CH1 domains), a Fv fragment (consisting of the VL and VH domains of a single arm of an antibody), a dAb fragment (consisting of a single variable domain fragment (VH or VL domain), a single chain Fv (scFv) comprising the two domains of a Fv fragment, VL and VH, that are fused together, and in some embodiments, with a linker to make a single protein chain. In some embodiments, the antibody is an scFv that specifically binds to human Siglec-5.

In some preferred embodiments, an anti-Siglec-5 antibody is provided that inhibits the interaction between Siglec-5 and a Siglec-5 ligand expressed on a cancer cell. In some embodiments, an anti-Siglec-5 antibody of the present disclosure decreases or reduces binding of recombinant human Siglec-5 to a cancer cell expressing a Siglec-5 ligand. In other embodiments, an anti-Siglec-5 antibody decreases binding of Siglec-5 to its cognate ligand expressed on a cancer cell without significantly decreasing or reducing cell surface levels of Siglec-5 expressed on an immune cell (e.g. activated T cell) in vitro and/or in vivo.

As used herein, an anti-Siglec-5 antibody inhibits the interaction (e.g., binding) between Siglec-5 and one or more Siglec-5 ligands if it decreases ligand binding to Siglec-5 by at least 20% at saturating antibody concentrations utilizing any in vitro assay or cell-based culture assay described herein or known in the art. In some aspects, an anti-Siglec-5 antibody inhibits the interaction between Siglec-5 and Siglec-5 ligand expressed on a cancer cell if it induces a decrease of 20% or more in binding of Siglec-5 to a cancer cell expressing a Siglec-5 ligand.

In some embodiments, anti-Siglec-5 antibodies of the present disclosure decrease binding of Siglec-5 to one or more Siglec-5 ligands by at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more as compared to binding of Siglec-5 in the absence of the anti-Siglec-5 antibody.

Additionally, anti-Siglec-5 antibodies of the present disclosure can be used to prevent, reduce the risk of, or treat cancer. In some embodiments, anti-Siglec-5 antibodies of the present disclosure can be used to prevent/reverse T cell inactivation caused by interaction of Siglec-5 (expressed on activated T cells) with its cognate ligand (expressed on cancer cells) in the tumor microenvironment in an individual in need thereof. In some embodiments, anti-Siglec-5 antibodies of the present disclosure are monoclonal antibodies. Thus, in some aspects, a method of reducing or inhibiting growth of a cancer or tumor cells (e.g., treating a cancer) in a subject is provided, the method comprising administering to the subject an anti-Siglec-5 antibody molecule described herein, e.g., a therapeutically effective amount of an anti-Siglec-5 antibody molecule, alone or in combination with a second agent, e.g., an immunomodulator (e.g., an anti-PD-1, PD-L1, LAG-3 or CEACAM-1 inhibitor (e.g., antibody), or a combination thereof.

In some aspects, an anti-Siglec-5 antibody of the present disclosure prevents a Siglec-5 ligand-mediated decrease in secretion of one or more proinflammatory cytokines (e.g., IFN-g and IL-22) and/or increase in secretion of one or more Th2 cytokines (e.g., IL-4, IL-5, and IL-13) by an activated T cell expressing Siglec-5 upon antigen (e.g., tumor antigen) stimulation.

In some aspects, an anti-Siglec-5 antibody of the present disclosure enhances secretion of one or more proinflammatory cytokines (e.g., IFN-g, TNF-alpha, and/or IL-22) and/or proliferation in T cells, e.g., CD4+ or CD8+ T cells, e.g. in CD4+ T cells that were stimulated with anti-CD3/CD28 in the presence of IL-12 or in T cell-DC autologous culture assays with anti-CD3/CD28 stimulation.

In some aspects, an anti-Siglec-5 antibody of the present disclosure enhances cytotoxic NK (natural killer) cell activity against a target cancer cell, e.g. in an in vitro assay. In other aspects, an anti-Siglec-5 antibody enhances capacity of macrophages or antigen presenting cells to stimulate a T cell response, e.g., increasing IL-12 secretion of antigen presenting cells.

In other aspects, an anti-Siglec-5 antibody binds specifically to an epitope of Siglec-5, e.g. the same or similar epitope as the epitope recognized by an antibody molecule as described herein.

In certain embodiments, an anti-Siglec-5 antibody that inhibits interaction between Siglec-5 and one or more Siglec-5 ligands is an anti-Siglec-5 antibody that binds or physically interacts with a Siglec-5. The anti-Siglec-5 antibody may have nanomolar or even picomolar affinities for the target antigen (e.g., Siglec-5). In certain embodiments, the Kd of the antibody is about 10 pM to about 100 nM. For example, Kd of the antibody is any of about 100 nM, about 50 nM, about 10 nM, about 1 nM, about 900 pM, about 800 pM, about 790 pM, about 780 pM, about 770 pM, about 760 pM, about 750 pM, about 740 pM, about 730 pM, about 720 pM, about 710 pM, about 700 pM, about 650 pM, about 600 pM, about 590 pM, about 580 pM, about 570 pM, about 560 pM, about 550 pM, about 540 pM, about 530 pM, about 520 pM, about 510 pM, about 500 pM, about 450 pM, about 400 pM, about 350 pM about 300 pM, about 290 pM, about 280 pM, about 270 pM, about 260 pM, about 250 pM, about 240 pM, about 230 pM, about 220 pM, about 210 pM, about 200 pM, about 150 pM, about 100 pM, about 50 pM, about 40 pM, about 30 pM, or about 20 pM, or about 15 pM to any of about 1 pM, about 2 pM, about 3 pM, about 4 pM, about 5 pM, about 6 pM, about 7 pM, about 8 pM, about 9 pM, about 10 pM, about 11 pM, about 12 pM, about 13 pM, or about 14 pM. Methods for the preparation and selection of antibodies that interact and/or bind with specificity to a Siglec-5 are described herein.

In some embodiments, an anti-Siglec-5 antibody of the present disclosure binds to a human Siglec-5. In some embodiments, an anti-Siglec-5 antibody of the present disclosure specifically binds to human Siglec-5. In some embodiments, an anti-Siglec-5 antibody of the present disclosure binds to Siglec-5 but does not bind to Siglec-14. In some embodiments, an anti-Siglec-5 antibody of the present disclosure binds human Siglec-5 but does not bind human Siglec-14. In some embodiments, an anti-Siglec-5 antibody of the present disclosure binds human Siglec-5 but does not bind cyno Siglec-5.

In some aspects, the present disclosure provides methods for detecting the presence of Siglec-5 a sample, e.g., in vitro or in vivo (e.g., a biological sample, e.g., blood, serum, semen or urine, or a tissue biopsy, e.g., from a hyperproliferative or cancerous lesion). The methods herein can be used to evaluate (e.g., monitor treatment or progression of, diagnose and/or stage a disorder described herein, e.g., an immune disorder, a cancer, or an infectious disease, in a subject). The method may include: (i) contacting the sample with (and optionally, a reference, e.g., a control sample), or administering to the subject, an anti-Siglec-5 antibody molecule as described herein, under conditions that allow interaction to occur, and (ii) detecting whether there is formation of a complex between the antibody molecule and the sample (and optionally, the reference, e.g., control, sample). Formation of the complex is indicative of the presence of Siglec-5, and can indicate the suitability or need for a treatment described herein. The method can involve, e.g., an immunohistochemistry, immunocytochemistry, flow cytometry, antibody molecule complexed magnetic beads, ELISA assays, PCR-techniques (e.g., RT-PCR).

Typically, the anti-Siglec-5 antibody molecule used in the in vivo and in vitro diagnostic methods is directly or indirectly labeled with a detectable substance to facilitate detection of the bound or unbound binding agent. Suitable detectable substances include various biologically active cozymes, prosthetic groups, fluorescent materials, luminescent materials, paramagnetic (e.g., nuclear magnetic resonance active) materials, and radioactive materials.

In some aspects, the present disclosure provides diagnostic or therapeutic kits that include the anti-Siglec-5 antibody molecules described herein and instructions for use.

In some aspects, the present disclosure provides a screening method for identifying a molecule that inhibits the interaction between Siglec-5 and a Siglec-5 ligand expressed on a cancer cell useful in the treatment methods described herein. In a preferred embodiment, the screening method identifies a small molecule that inhibits the interaction between Siglec-5 and a Siglec-5 ligand expressed on a cancer cell.

In some preferred embodiments, in such a screening assay, a first binding mixture is formed by combining Siglec-5 and an antibody of the invention; and the amount of binding in the first binding mixture (M₀) is measured. A second binding mixture is also formed by combining Siglec-5, the antibody, and the compound or agent to be screened, and the amount of binding in the second binding mixture (M₁) is measured. A compound to be tested may be another anti-Siglec-5 antibody or a small molecule. The amounts of binding in the first and second binding mixtures are then compared, for example, by calculating the M₁/M₀ ratio. The compound or agent is considered to be capable of modulating a Siglec-5-associated downregulation of immune responses if a decrease in binding in the second binding mixture as compared to the first binding mixture is observed. The formulation and optimization of binding mixtures is within the level of skill in the art, such binding mixtures may also contain buffers and salts necessary to enhance or to optimize binding, and additional control assays may be included in the screening assay of the invention. Compounds found to reduce the Siglec-5-antibody binding by at least about 10% (i.e., M₁/M₀<0.9), preferably greater than about 30% may thus be identified and then, if desired, secondarily screened for the capacity to ameliorate a disorder in other assays or in a relevant animal model of disease. The strength of the binding between Siglec-5 and an antibody can be measured using, for example, an enzyme-linked immunoadsorption assay (ELISA), radio-immunoassay (RIA), surface plasmon resonance-based technology (e.g., Biacore), all of which are techniques well known in the art.

In related embodiments, small molecule candidates can be generated and screened for their ability to inhibit the interaction between Siglec-5 and a Siglec-5 ligand expressed on a cancer cell using high-throughput synthesis of a sialoside analog library using click chemistry, coupled with microarray technology to identify hits for human Siglec-5, e.g. according to the methods of Rillahan et al., Angew Chem Int Ed Engl., 51(44):11014-11018 (2012). Such compounds can then be secondarily screened for the capacity to stimulate T cells in an MLR or other in vitro assays or in a relevant animal model of disease (e.g., cancer).

In yet other embodiments, a method for downregulating an immune response is provided comprising administering to a subject an anti-Siglec-5 antibody that decreases a T cell response in the subject. Anti-Siglec-5 antibodies useful for downregulating an immune response may act as Siglec-5 agonists and have the property of enhancing Siglec-5 mediated attenuation of the immune response. Such antibodies can be identified by their effect on reducing immune cell (e.g. T cell) secretion of one or more pro-inflammatory cytokines (e.g., IFNγ, IL-2, IL-1a, IL-1b, TNF-α, IL-8) and/or increasing secretion on one or more Th2 cytokines (e.g., IL-4 and/or IL-10) and/or reducing proliferation of T cells and are useful in treating and/or preventing autoimmune diseases (such as rheumatoid arthritis, psoriasis, psoriatic arthritis, ankylosing spondylitis, type 1 diabetes, multiple sclerosis, inflammatory bowel disease, Crohn's disease, systemic lupus erythematosus and asthma), hyperproliferative immune disorders, tissue, skin and organ transplant rejection and graft-versus-host disease (GVHD).

### DESCRIPTION OF THE DRAWINGS

FIG. 1 illustrates binding of anti-Siglec-5 monoclonal antibodies (mAbs) to Siglec-5 expressed on the surface of activated human T cells.

FIG. 2 illustrates the effect of anti-Siglec-5 antibodies on activated T cell populations following 4 days of PBMC stimulation in the presence of the mAbs. The data is an average of two technical repeats from a single donor (donor 1). PD-1 and BL are mAbs from Biolegend (BL) that bind the PD-1 or Siglec-5 receptors, respectively. IgG is a negative control where only IgG is added, Cell counts were normalized to the IgG sample control.

FIG. 3 illustrates the effect of anti-Siglec-5 antibodies on IL-17A following 4 days of stimulation in the presence of the mAbs. IgG served as a negative control. Murine anti-PD-1 antibody served as a positive control. A commercial anti-Siglec-5 antibody (“BL”) was included in the study as well (Biolegend). The data is an average of two technical repeats from a single donor (donor 1).

FIG. 4 illustrates the effect of anti-Siglec-5 antibodies on IT-2 following 4 days of PBMC stimulation in the presence of the mAbs. IgG served as a negative control. Murine anti-PD-1 antibody served as a positive control. A commercial anti-Siglec-5 antibody (“BL”) was included in the study as well. The data is an average of two technical repeats from a single donor (donor 1).

FIG. 5 illustrates the effect of anti-Siglec-5 antibodies on IL-6 following 4 days of PBMC stimulation in the presence of the mAbs. IgG served as a negative control. Murine anti-PD-1 antibody served as a positive control. The data is an average of two technical repeats from a single donor (donor 1).

FIG. 6 illustrates the effect of anti-Siglec-5 antibodies on IFN-y production following stimulation of engineered human T cells transduced with a TCR (13831) specific for melanoma antigen tyrosinase with melanoma MEL624-derived antigen. A murine anti-PD-1 immune checkpoint inhibitor antibody and soluble Siglec-5 fusion protein (Siglec-5 Fc) served as a positive controls; IgG served as a negative control. The data is an average of two technical repeats from a single donor (donor 2).

FIG. 7 illustrates the effect of anti-Siglec-5 antibodies on IL-5 production following stimulation of engineered human T cells transduced with a TCR (13831) specific for melanoma antigen tyrosinase with melanoma MEL624-derived antigen. A murine anti-PD-1 immune checkpoint inhibitor antibody and soluble Siglec-5 fusion protein (Siglec-5 Fc) served as a positive controls; IgG served as a negative control. The data is an average of two technical repeats from a single donor (donor 2).

FIG. 8 the effect of anti-Siglec-5 antibodies on IL-13 production following stimulation of engineered human T cells transduced with a TCR (13831) specific for melanoma antigen tyrosinase with melanoma MEL624-derived antigen. A murine anti-PD-1 immune checkpoint inhibitor antibody and soluble Siglec-5 fusion protein (Siglec-5 Fc) served as a positive controls; IgG served as a negative control. The data is an average of two technical repeats from a single donor (donor 2).

### DETAILED DESCRIPTION

### Definitions

The term “antibody” as referred to herein includes whole antibodies and any antigen-binding fragment (i.e., “antigen-binding portion”) or single chains thereof. An “antibody” refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as Vn) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., Siglec-5). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the Vi and Vu regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds Siglec-5 is substantially free of antibodies that specifically bind antigens other than Siglec-5). An isolated antibody that specifically binds Siglec-5 may, however, have cross-reactivity to other antigens, such as Siglec-5 molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., Ig or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human Siglec-5” is intended to refer to an antibody that binds to human Siglec-5 with a K_{D} of 1×10⁻⁷ M or less, more preferably 5×10⁻⁸ M or less, more preferably 1×10⁻⁸ M or less, more preferably 5×10⁻⁹ M or less.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} an antibody is by using surface plasmon resonance, preferably using a biosensor system such as a Biacore® system.

As used herein, the term “high affinity” for an IgG antibody refers to an antibody having a K_{D} of 10⁻⁸ M or less, more preferably 10⁻⁹ M or less and even more preferably 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, more preferably 10⁻⁸ M or less, even more preferably 10⁻⁹ M or less.

As used herein, the terms “interfering RNA” and “interfering RNA molecule” refer to all RNA or RNA-like molecules that can interact with RISC and participate in RISC-mediated changes in gene expression and preferably reduce (i.e., knock-down) the translation of specific messenger RNAs (mRNAs) such as a Siglec-5 mRNA. Examples of interfering RNA molecules that can interact with RISC include short hairpin RNAs (shRNAs), single-stranded siRNAs, microRNAs (miRNAs), picoRNAs (piRNAs), and dicer-substrate 27-mer duplexes. Examples of “RNA-like” molecules that can interact with RISC include siRNA, single-stranded siRNA, miRNA, piRNA, asymmetrical siRNA, and shRNA molecules that contain one or more chemically modified nucleotides, one or more non-nucleotides, one or more deoxyribonucleotides, and/or one or more non-phosphodiester linkages. Thus, siRNAs, single-stranded siRNAs, shRNAs, miRNAs, piRNA, asymmetrical siRNA, and dicer-substrate 27-mer duplexes are subsets of “interfering RNAs” or “interfering RNA molecules.

The term “siRNA” as used herein refers to a double-stranded interfering RNA unless otherwise noted. Typically, an siRNA used in a method of the invention is a double-stranded nucleic acid molecule comprising two nucleotide strands, each strand having about 19 to about 28 nucleotides (i.e. about 19, 20, 21, 22, 23, 24, 25, 26, 27, or 28 nucleotides). Typically, an interfering RNA used in a method of the invention has a length of about 19 to 49 nucleotides. The phrase “length of 19 to 49 nucleotides” when referring to a double-stranded interfering RNA means that the antisense and sense strands independently have a length of about 19 to about 49 nucleotides, including interfering RNA molecules where the sense and antisense strands are connected by a linker molecule.

The term “treatment” or “therapy” refers to administering an active agent with the purpose to cure, heal, alleviate, relieve, alter, remedy, ameliorate, improve, or affect a condition (e.g., a disease), the symptoms of the condition, or to prevent or delay the onset of the symptoms, complications, biochemical indicia of a disease, or otherwise arrest or inhibit further development of the disease, condition, or disorder in a statistically significant manner.

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.geg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. (See www.nebi.nlm.nih.gov).

Siglec-5 is a type-1 transmembrane protein that belongs to the immunoglobulin superfamily. The single transmembrane domain of Siglec-5 links 4 extracellular IG-like domains to an 89 amino acid long cytoplasmic tail that has two tyrosine-based conserved motifs. The amino acid sequence of a human Siglec-5 is provided below:

```
(SEQ ID NO: 1)
Met Leu Pro Leu Leu Leu Leu Pro Leu Leu Trp Gly Gly Ser Leu Gln
1 5 10 15
Glu Lys Pro Val Tyr Glu Leu Gln Val Gln Lys Ser Val Thr Val Gln
                        20 25 30
Glu Gly Leu Cys Val Leu Val Pro Cys Ser Phe Ser Tyr Pro Trp Arg
                35 40 45
Ser Trp Tyr Ser Ser Pro Pro Leu Tyr Val Tyr Trp Phe Arg Asp Gly
        50 55 60
Glu Ile Pro Tyr Tyr Ala Glu Val Val Ala Thr Asn Asn Pro Asp Arg
65 70 75 80
Arg Val Lys Pro Glu Thr Gln Gly Arg Phe Arg Leu Leu Gly Asp Val
                                85 90 95
Gln Lys Lys Asn Cys Ser Leu Ser Ile Gly Asp Ala Arg Met Glu Asp
                        100 105 110
Thr Gly Ser Tyr Phe Phe Arg Val Glu Arg Gly Arg Asp Val Lys Tyr
                115 120 125
Ser Tyr Gln Gln Asn Lys Leu Asn Leu Glu Val Thr Ala Leu Ile Glu
        130 135 140
Lys Pro Asp Ile His Phe Leu Glu Pro Leu Glu Ser Gly Arg Pro Thr
145 150 155 160
Arg Leu Ser Cys Ser Leu Pro Gly Ser Cys Glu Ala Gly Pro Pro Leu
                                165 170 175
Thr Phe Ser Trp Thr Gly Asn Ala Leu Ser Pro Leu Asp Pro Glu Thr
                        180 185 190
Thr Arg Ser Ser Glu Leu Thr Leu Thr Pro Arg Pro Glu Asp His Gly
                195 200 205
Thr Asn Leu Thr Cys Gln Met Lys Arg Gln Gly Ala Gln Val Thr Thr
        210 215 220
Glu Arg Thr Val Gln Leu Asn Val Ser Tyr Ala Pro Gln Thr Ile Thr
225 230 235 240
Ile Phe Arg Asn Gly Ile Ala Leu Glu Ile Leu Gln Asn Thr Ser Tyr
                                245 250 255
Leu Pro Val Leu Glu Gly Gln Ala Leu Arg Leu Leu Cys Asp Ala Pro
                        260 265 270
Ser Asn Pro Pro Ala His Leu Ser Trp Phe Gln Gly Ser Pro Ala Leu
                275 280 285
Asn Ala Thr Pro Ile Ser Asn Thr Gly Ile Leu Glu Leu Arg Arg Val
        290 295 300
Arg Ser Ala Glu Glu Gly Gly Phe Thr Cys Arg Ala Gln His Pro Leu
305 310 315 320
Gly Phe Leu Gln Ile Phe Leu Asn Leu Ser Val Tyr Ser Leu Pro Gln
                                325 330 335
Leu Leu Gly Pro Ser Cys Ser Trp Glu Ala Glu Gly Leu His Cys Arg
                        340 345 350
Cys Ser Phe Arg Ala Arg Pro Ala Pro Ser Leu Cys Trp Arg Leu Glu
                355 360 365
Glu Lys Pro Leu Glu Gly Asn Ser Ser Gln Gly Ser Phe Lys Val Asn
        370 375 380
Ser Ser Ser Ala Gly Pro Trp Ala Asn Ser Ser Leu Ile Leu His Gly
385 390 395 400
Gly Leu Ser Ser Asp Leu Lys Val Ser Cys Lys Ala Trp Asn Ile Tyr
                                405 410 415
Gly Ser Gln Ser Gly Ser Val Leu Leu Leu Gln Gly Arg Ser Asn Leu
                        420 425 430
Gly Thr Gly Val Val Pro Ala Ala Leu Gly Gly Ala Gly Val Met Ala
                435 440 445
Leu Leu Cys Ile Cys Leu Cys Leu Ile Phe Phe Leu Ile Val Lys Ala
        450 455 460
Arg Arg Lys Gln Ala Ala Gly Arg Pro Glu Lys Met Asp Asp Glu Asp
465 470 475 480
Pro Ile Met Gly Thr Ile Thr Ser Gly Ser Arg Lys Lys Pro Trp Pro
                                485 490 495
Asp Ser Pro Gly Asp Gln Ala Ser Pro Pro Gly Asp Ala Pro Pro Leu
                        500 505 510
Glu Glu Gin Lys Glu Leu His Tyr Ala Ser Leu Ser Phe Ser Glu Met
                515 520 525
Lys Ser Arg Glu Pro Lys Asp Gln Glu Ala Pro Ser Thr Thr Glu Tyr
        530 535 540
Ser Glu Ile Lys Thr Ser Lys
545 550
```

Siglec-5 proteins, such as human Siglec-5, contain several domains, including without limitation, a signal sequence located at amino acid residues 1-16 of SEQ ID NO: 1, an extracellular immunoglobulin-like variable-type (IgV) domain located at amino acid residues 19-136 of SEQ ID NO: 1, two Ig-like C2-type domains located at amino acid residues 146-229 and 236-330 of SEQ ID NO: 1, a transmembrane domain located at amino acid residues 442-462 of SEQ ID NO: 1, an ITIM motif 1 located at amino acid residues 518-523 of SEQ ID NO: 1, and a SLAM-like motif located at amino acid residues 542-547 of SEQ ID NO: 1. As one of skill in the art will appreciate, the beginning and ending residues of the domains of the present disclosure may vary depending upon the computer modeling program used or the method used for determining the domain.

As used herein a “Siglec-5” protein of the present disclosure includes, without limitation, a mammalian Siglec-5 protein, human Siglec-5 protein, and primate Siglec-5 protein. Additionally, anti-Siglec-5 antibodies of the present disclosure may bind an epitope within one or more of a mammalian Siglec-5 protein, human Siglec-5 protein, and primate Siglec-5. In some embodiments, anti-Siglec-5 antibodies of the present disclosure may bind specifically to a human Siglec-5 protein. In preferred embodiments, an anti-Siglec-5 antibody binds to an epitope within amino acid residues 19-136, within 146-229 or within 236-330 of human Siglec-5 protein.

Siglec-5 binds to both a2,3- and a2,6 linked, and to a lesser extent, a2,8-linked sialic acids. Several protein ligands of Siglec-5 have been identified including b-protein (expressed in Group B Streptococcus), HSP70 and PSLG-1. Siglec-5 is identified herein as an inhibitory receptor in activated T cells, with engagement of Siglec-5 with its cognate ligand on a tumor cell suppressing T cell-specific anti-tumor response. As such, the present application provides methods for reversing T cell exhaustion by blocking the interaction between Siglec-5 and a Siglec-5 ligand expressed on a cancer cell.

The present application identifies Siglec-5 as having significant immune checkpoint qualities. Other Siglec family members may also have immune checkpoint qualities. As such, anti-Siglec-5 antibodies are provided which block the interaction between Siglec-5 and a Siglec-5 ligand expressed on a cancer cell and the therapeutic use of these antibodies as a checkpoint inhibitor. In some aspects, the antibodies are administered to reverse T-cell exhaustion and increase the effectiveness of CAR-T therapies. In other aspects, the antibodies are co-administered with one or more other checkpoint inhibitors such as Keytruda and other therapies that target PD-1/PDL-1 pathway, In some aspects, co-administration of the anti-Siglec-5 antibody and the one or more other checkpoint inhibitors exhibit synergistic antitumor effects. In some preferred embodiments, an anti-Siglec-5 antibody as herein described is administered in combination with (i) a checkpoint inhibitor targeting programmed cell death receptor-1 (PD-1) such as, but not limited to, nivolumab, pembrolizumab, cemiplimab; (ii) a checkpoint inhibitor targeting programmed cell death ligand-1 (PD-L1) such as, but not limited to, atezolizumab, avelumab, durvalumab; (iii) a checkpoint inhibitor targeting cytotoxic T lymphocyte-associated molecule-4 (CTLA-4) such as, but not limited to, Ipilimumab; (iv) a checkpoint inhibitor targeting Lymphocyte activation gene-3 (LAG-3 also known as CD223); (v) a checkpoint inhibitor targeting TIM-3, (vi) a checkpoint inhibitor targeting B7-H3 or B7-H4 and/or (vii) a checkpoint inhibitor targeting A2aR, CD73, NKG2A, and/or PVRIGPVRL2; and/or pathways that act on other inhibitory immune mechanisms such as, but not limited to, CEACAM1, CEACAM5, CEACAM6, FAK, CCL2/CCR2, LIF, CD47SIRPα, CSF-1(M-CSF), IL-1, IL-1R3(IL-1RAP), IL-8, SemaphorinsSEMA4D, Ang2,EVER-1, Ax1, and/or Phosphatidylserine,

In some embodiments, the anti-Siglec-5 antibody molecule is administered concurrently, simultaneously or sequentially with the one or more additional immune checkpoint inhibitors, Concurrent administration means that the administration regimens of two separate therapeutic agents overlap by at least one day.

In a related aspect, the anti-Siglec-5 antibody molecule is administered in combination with an anti-TIM-3 agent, including, without limitation, MBG453, Sym023 and/or TSR-022 for the treatment and/or prevention of cancer. In some embodiments, the anti-Siglec-5 molecule is administered in combination with an anti-Tim-3 antibody or antigen-binding fragment thereof.

In a preferred aspect, the anti-Siglec-5 antibody molecule is administered in combination with an anti-PD-1 agent for the treatment and/or prevention of cancer. In some embodiments, the anti-PD-1 agent is an antibody or antigen-binding fragment thereof, representative examples of which include Cemiplimab, Nivolumab, and Pembrolizumab.

In another aspect, the anti-Siglec-5 antibody molecule is administered in combination with an anti-PD-L1 agent for the treatment and/or prevention of cancer. In some embodiments, the anti-PD-L1 agent is an antibody or antigen-binding fragment thereof, representative examples of which include Atezolizumab, Avelumab, and Durvalumab.

In another aspect, the anti-Siglec-5 antibody molecule is administered in combination with an anti-CTLA-4 agent for the treatment and/or prevention of cancer. In some embodiments, the anti-CTLA-4 agent is an antibody or antigen-binding fragment thereof, a representative example of which is Ipilimumab.

In another aspect, the anti-Siglec-5 antibody molecule is administered in combination with an anti-LAG-3 agent such as LAG525 (IMP701), REGN3767 (R3767), BI 754,091, IMP321 or FS118 for the treatment and/or prevention of cancer. In some embodiments, the anti-Lag3 agent is antibody or antigen-binding fragment thereof, a representative example of which is tebotelimab.

In another aspect, the anti-Siglec-5 antibody molecule is administered in combination with an anti-B7-H3 or anti-B7-H4 agent such as MGC018 or FPA150 for the treatment and/or prevention of cancer. In some embodiments, the anti-B7-H3 or anti-B7-H4 agent is an antibody or antigen-binding fragment thereof.

In other embodiments, the anti-Siglec-5 antibody molecule is administered in combination with a modulator, e.g., agonist, of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CD8, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, or CD83 ligand,

In a preferred aspect, the anti-Siglec-5 antibody molecule is administered in combination with an OX40 agonist for the treatment and/or prevention of cancer. In some embodiments, the OX40 agonist is an antibody or antigen-binding fragment thereof, representative examples of which include tislezliumab, HFB301001, YH002, ivuxolimab (PF-04518600), and INBRX-106.

These combinations or cocktails of medications may act additively or synergistically in the treatment and/or prevention of cancer.

Siglec-5 and other Siglec-5 family members (Siglec 1, 2, 3, 4, 5, 6, 7, 8, 9, 10, 11, 12, 13, 14, 15) are identified herein as checkpoint protein(s). As such, antibodies or other compounds that block the interaction between Siglec-5 and a Siglec-5 ligand expressed on a cancer cell act as checkpoint inhibitors of these targets. These checkpoint inhibitor(s) may be administered alone or in combination with other checkpoint inhibitor(s) (and other therapies) (antibody-therapeutics [Keytruda]) to reduce tumor size in both liquid (lymphoma) and solid tumors in a subject (e.g., a mammal). These checkpoint inhibitor(s) may be administered alone or in combination with other therapies to reduce and reverse t-cell exhaustion, in the treatment of liquid (lymphoma) or solid tumors. These checkpoint inhibitor(s) may also be administered alone or in combination with other checkpoint inhibitors and therapies to reduce and reverse T-cell exhaustion in CAR-T therapies. The checkpoint inhibitor(s) act to reverse immune system exhaustion in the tumor microenvironment.

In any of the above-described embodiments, the antibody or antibody fragment of the invention may bind human SIGLEC5 with a kassoc of about 7.5×10⁻⁵ 1/M·s or faster. In one embodiment, the antibody or antibody fragment may bind human SIGLEC5 with a kassoc of about 1×10⁻⁶ 1/M·s or faster.

In any of the above-described embodiments, the antibody or antibody fragment may bind human SIGLEC5 with a kdissoc of about 2×10⁻⁵ 1/s or slower. In one embodiment, the antibody or antibody fragment may bind human SIGLEC5 with a kdissoc of about 2.7×10⁻⁵ 1/s or slower. In one embodiment, the antibody or antibody fragment may bind human SIGLEC5 with a kdissoc of about 3×10⁻⁵ 1/s or slower.

KD, kassoc and kdissoc values can be measured using any available method. In preferred embodiments, the disassociation constant is measured using bio-light interferometry (for example, the ForteBio Octet method). In other preferred embodiments, the disassociation constant can be measured using surface plasmon resonance (e.g., Biacore) or Kinexa.

Further, in any of the above-described embodiments, the antibody or antibody fragment of the invention may block binding of human PSGL1 to human SIGLEC5 with an IC50 of about 1 nM or lower. The blockade of ligand binding can be measured and the IC50 calculated using any method known in the art, for example, the FACS or FMAT methods described in the Examples herein.

The invention also comprises an antibody or antibody fragment which competes for a binding epitope on human SIGLEC5 with any of the antibodies described above, and which blocks the binding of human PSGL1 or to human SIGLEC5 with an IC50 of about 1 nM or lower.

In some embodiments, the antibody or antibody fragments of the invention are chimeric antibodies or fragments of chimeric antibodies.

In some embodiments, the antibody or antibody fragments of the invention are human antibodies or fragments of human antibodies.

In some embodiments, the antibody or antibody fragments of the invention are humanized antibodies or fragments of humanized antibodies.

In some embodiments, the antibody fragments of the invention are Fab, Fab′, Fab′-SH, Fv, scFv, or F(ab′)2 antibody fragments.

In some embodiments, the antibody fragments of the invention are diabodies.

The invention also comprises bispecific antibodies comprising any one of the antibody or antibody fragments described above that bind to human SIGLEC5.

In some embodiments, the isolated anti-SIGLEC5 antibodies and antibody fragments of the invention increase T cell activation as measured by typical means known to one skilled in the art (including, without limitation, increased immune cell proliferation, increased cytokine secretion or expression of activation markers such as CD25 and/or CD69).

In any of the above-described embodiments, the antibody or antibody fragment of the invention may enhance the immune response after stimulation with Staphylococcus Enterotoxin B or Tetanus Toxoid ex vivo or in vivo. The increased immune activation may be determined using methods known to anyone skilled in the art, for example, quantifying proliferation of immune cells (such as T cells) or cytokine production by immune cells (for example production of IFNγ or IL-2 by T cells).

The invention also comprises a method of increasing the activity, or reducing the downmodulation, of an immune cell comprising contacting the immune cell with any one of the antibodies or antibody fragments of the invention. This method could be used to treat cancer or infectious diseases (such as chronic viral infections), or could be used as an adjuvant to a prophylactic or therapeutic vaccine.

The invention also comprises a method of increasing an immune response to an antigen, comprising contacting an immune cell with an antigen and an anti-SIGLEC5 antibody or an antibody fragment such that an immune response to the antigen is increased or enhanced. This method could be conducted in vivo (in a subject) or ex vivo.

In some embodiments, an anti-SIGLEC5 antibody or antibody fragment may be combined with a second therapeutic agent or treatment modality. In one embodiment, an anti-SIGLEC5 antibody or antibody fragment may be combined with cancer treatments involving the application of recombinant cytokines or secreted immune factors. Non-limiting examples of combinations include combining anti-SIGLEC5 antibody with recombinant IL-2 or recombinant IFNα2 for the treatment of melanoma or renal cell carcinoma. Recombinant IL-2 enhances T cell outgrowth in cancer patients. Recombinant IFNα2 inhibits cancer cell growth but also increases expression of the inhibitory ligands for SIGLEC5 on cancer cells, antigen-presenting cells and other somatic cells in the treated patients. Anti-SIGLEC5 can be combined with other cytokines that might be considered useful for the treatment of cancer or infectious diseases.

In some embodiments, anti-SIGLEC5 antibodies or antibody fragments can be combined with a vaccine to prevent or treat cancer or infectious disease. As a non-limiting example, anti-SIGLEC5 could be combined with a protein, peptide or DNA vaccine containing one or more antigens which are relevant to the cancer or infection to be treated, or a vaccine comprising of dendritic cells pulsed with such an antigen. Another embodiment includes the use of anti-SIGLEC5 with (attenuated) cancer cell or whole virus vaccines. One embodiment involves a combination of anti-SIGLEC5 therapy with a whole cell cancer vaccine that is engineered to secrete GM-CSF.

In some embodiments, anti-SIGLEC5 antibodies or antibody fragments can be combined with treatment that is considered to be standard of care in cancer or infectious disease. Rationale for such combinations is that concurrent increased immune activation by anti-SIGLEC5 will induce or facilitate initial clinical response to standard of care treatment, induce durable clinical response and long-term immune control of disease.

In one embodiment, treatment with anti-SIGLEC5 antibodies or antibody fragments may be combined with chemotherapy. Chemotherapy using cytotoxic agents will result in cancer cell death thereby increasing release of tumor antigens. Such increased availability of tumor antigen may result in synergy with anti-SIGLEC5 treatment. A non-limiting example is provided by the use of decarbazine or temozolomide for the treatment of melanoma and gemcitabine for pancreatic cancer.

In one embodiment, treatment with anti-SIGLEC5 antibodies or antibody fragments may be combined with radiotherapy. Radiotherapy induces cancer cell death and increasing availability of tumor antigens for presentation and activation of immune cells.

In another embodiment, treatment with anti-SIGLEC5 antibodies or antibody fragments may be combined with surgery, to remove cancer cells from a subject.

In other embodiments, anti-SIGLEC5 antibodies or antibody fragments may be combined with therapies which may result in synergy with SIGLEC5 blockade including targeted agents used for hormone deprivation or inhibition of angiogenesis, or targeting proteins active in tumor cells, all resulting in enhanced tumor cell death and availability of immune stimulating tumor antigens. In combination with an anti-SIGLEC5 antibody or antibody fragment, increased T cell activation may result in durable immune control of cancer.

In some embodiments, an anti-SIGLEC5 antibody or antibody fragment may be combined with another therapeutic antibody useful for the treatment of cancer or infectious disease. A non-limiting example is provided by the combination of anti-SIGLEC5 with an antibody targeting Her2/neu or targeting the EGF receptor. In another non-limiting example, an anti-SIGLEC5 antibody or antibody fragment is combined with treatment targeting VEGF or its receptors. In another embodiment, an anti-SIGLEC5 antibody or antibody fragment is combined with anti-CTLA-4. In yet another nonlimiting example, anti-SIGLEC5 is combined with an antibody that targets RSV.

Monoclonal antibodies (mAbs) of the present invention can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

A preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g. U.S. Pat. No. 5,225,539 to Winter, and U.S. Pat. Nos. 5,530,101; 5,585,089; 5,693,762 and 6,180,370 to Queen et al.).

In some preferred embodiments, anti-Siglec-5 antibodies are generated according to the methods described in U.S. Pat. No. 7,731,969, the entire contents of which are incorporated herein by reference. In particularly preferred embodiments, the antibodies are generated according to a method comprising a) immunizing an animal with a human Siglec-5 antigen and isolating splenocytes from the animal; b) depleting the isolated splenocytes of CD138-positive cells to obtain depleted immunized cells; c) contacting the depleted immunized cells with an activating agent to obtain activated, antigen-specific B-lymphocytes; d) immortalizing the activated, antigen-specific B-lymphocytes, thereby producing immortalized human Siglec-5-specific plasma cells; and e) growing the immortalized Siglec-5-specific plasma cells to obtain antibodies that specifically bind to Siglec-5.

In some embodiments, a nucleic acid molecule encoding an anti-Siglec-5 antibody as herein described is provided. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of the invention can be, for example, DNA or RNA and may or may not contain intronic sequences. In a preferred embodiment, the nucleic acid is a cDNA molecule.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Some embodiments of the invention are exemplified in the following items 1 to:
1. A method of immunomodulating using a Siglec to affect cancer cells.
2. A method of immunomodulating using a Siglec to affect immune cells in vitro.
3. A method of immunomodulating using a Siglec to affect immune cells in vivo in the organism.
4. A method of immunomodulating using a Siglec to affect T-cells.
5. A method of immunomodulating using a Siglec to affect immune cells selected from the group consisting of CAR-T cells (Chimeric Antigen Receptors) expressed in a T cell or NK (natural killer) cell or other immune cell type.
6. A method of immunomodulating using a Siglec to affect cancer cells in vitro.
7. A method of immunomodulating using a Siglec to affect cancer cells in vitro, killing them.
8. A method of immunomodulating using a Siglec to affect cancer cells in vivo in an organism.
9. A method of immunomodulating using a Siglec to affect cancer cells in vivo in an organism by killing them.
10. A method of immunomodulating using both Siglec family members (the receptors) and their ligands.
11. A composition of matter that can modulate individual receptors in the Siglec family of receptors and their ligands, wherein the composition includes an antibody that binds a member of the Siglec family.
12. A composition of matter that can modulate individual receptors in the Siglec family of receptors and their ligands, wherein the composition includes an antibody that binds to at least one of Siglec-5 and Siglec-14.
13, A method of treating an individual by using any of the preceding compositions,
14. A method of treating an individual by using any of the preceding compositions to treat a disease of the immune system.
15. A method of treating an individual by using any of the preceding compositions to treat a cancer.
16. A method of treating an individual using any of the preceding compositions by combining the composition with at least one other drug.
17. The method of item 16, wherein the at least one other drug is at least one checkpoint inhibitor.
18. The method of item 17, wherein the at least one checkpoint inhibitor is a checkpoint inhibitor targeting programmed cell death receptor-1 (PD-1).
19. The method of item 17, wherein the at least one checkpoint inhibitor is selected from the group consisting of nivolumab, pembrolizumab, and cemiplimab.
20. The method of item 17, wherein the at least one checkpoint inhibitor is a checkpoint inhibitor programmed cell death ligand-1 (PD-L1).
21. The method of item 17, wherein the at least one checkpoint inhibitor is selected from the group consisting of atezolizumab, avelumab, and durvalumab,
22. The method of item 17, wherein the at least one checkpoint inhibitor is a checkpoint inhibitor targeting cytotoxic T lymphocyte-associated molecule-4 (CTLA-4).
23. The method of item 17, wherein the at least one checkpoint inhibitor is Ipilimumab.
24. The method of item 17, wherein the at least one checkpoint inhibitor is a checkpoint inhibitor targeting Lymphocyte activation gene-3 (LAG-3 also known as CD223).
25, The method of item 17, wherein the at least one checkpoint inhibitor is a checkpoint inhibitor targeting TIM3.
26. The method of item 17, wherein the at least one checkpoint inhibitor is a checkpoint inhibitor targeting B7-H3.
27. The method of item 17, wherein the at least one checkpoint inhibitor is a checkpoint inhibitor targeting B7-H4.
28. The method of item 17, wherein the at least one checkpoint inhibitor is a checkpoint inhibitor targeting A2aR.
29. The method of item 17, wherein the at least one checkpoint inhibitor is a checkpoint inhibitor targeting CD73.
30. The method of item 17, wherein the at least one checkpoint inhibitor is a checkpoint inhibitor targeting NKG2A.
31. The method of item 17, wherein the at least one checkpoint inhibitor is a checkpoint inhibitor targeting PVRIGPVRL2.
32. The method of item 17, wherein the at least one checkpoint inhibitor is a checkpoint inhibitor targeting pathways that act on other inhibitory immune mechanisms.
33. The method of item 17, wherein the at least one checkpoint inhibitor is selected from the group consisting of CEACAM1, CEACAM5, CEACAM6, FAK, CCL2/CCR2, LIF, CD47SIRPα, CSF-1(M-CSF), IL-1, IL-1R3(IL-1RAP), IL-8, SemaphorinsSEMA4D, Ang2,EVER-1, Ax1, and Phosphatidylserine.
34. A method of treating an individual by using any of the preceding compositions in combination with CAR-T.
35. A method of treating an individual by using any of the preceding compositions in combination with CAR-T and at least one checkpoint inhibitor.
36. A method of treating an individual by using any of the preceding compositions in combination with CAR-T treatment in solid tumors.

### EXAMPLES

### Example 1—Generation of Anti-Siglec-5 Antibodies

### Immunization

An exemplary immunization protocol is illustrated below:

| Immunization (per | | | |
|---|---|---|---|
| mouse) | Day | Protein | Adjuvants |
| Initial | 0 | 100 mg | Freund's Complete |
| First boost | 14 | 100 mg | Freund's |
| | | | Incomplete |
| Second boost | 28 | 100 mg | Freund's |
| | | | Incomplete |
| Final boost | 42-59 | 100 mg | PBS |

Antibodies against human Siglec-5 were generated according to the methods described in U.S. Pat. No. 7,731,969, the entire contents of which are incorporated herein by reference.

Five 30-to 50-day old female BALB/c mice were injected intraperitoneally on days 0, 14, 21, 28, 35,42, 43, and 44 with a human Siglec-5 antigen (Cat #SI5-H5250 ACROBiosystems Inc.) or a soluble Siglec-5 generated internally, the antigen was mixed in alum adjuvant at 100 micrograms of protein antigen per mouse (total volume 100 micrograms/microliter per mouse). All mice were given a final intravenous boost by tail vein on days 43, 44, and 45. Alternatively, the mice can be rested and receive their final boost on days 57,58, and 59. On day 46 or day 60 respectively, the mice were euthanized for fusion. The spleens were harvested and lymphocytes isolated from the spleen.

In general, recombinant Siglec-5 protein can used as a standard antigen for primary mouse immunizations and mice can be immunized with Siglec-5 by intraperitoneal and subcutaneous injections with 50% of the protein injected at each location. Adjuvants such as Freund's or Alum are used. Alum was used exclusively when immunizing with Siglec-5.

### Spleen Recovery and Single Cell Suspension

Spleens from immunized mice were harvested one day after the last of three final boosts. Single-cell suspensions were made by perfusing the spleens with 20 mL of media per spleen. Red blood cells were lysed using Red Blood Cell Lysing Buffer (Sigma, St. Louis, Mo.) before resuspending in the appropriate cell media.

Cell counting

Cell recovery and viability following splenocyte isolation, cell sorting, development was assessed using an automated ViCell.TM. XR Cell Viability Analyzer system (Beckman Coulter, Inc., Fullerton, Calif.).

### Fusion

Lymphocytes were isolated from the spleens of the five mice as a single cell suspension, and then fused with murine SP2/0 myeloma cells in the presence of poly-ethylene glycol (50% PEG (Sigma, #P-7181). Fused cells were cultured using Azaserine (Sigma, #A1164-0.5 MG) medium selection. All culture wells contained 50,000 feeder cells. Feeder cells were produced by making a single cell suspension from the spleen of a naïve mouse. Fourteen days after the fusion, 1920 wells were tested for antigen specific responses.

### Hybridoma Solutions:

Hybridoma solutions comprised 95-100% Ethanol, 70% Ethanol Spray Bottle, 50% PEG (Sigma, #P-7181), Master OPI-HT Hybridoma Growth Media, HBSS (1×), [Sterile 1× PBS can be used as a substitute] Azaserine (50×) [Sigma, #A1164-0.5 MG or Fisher ICN15480510]

### OPI-HT Hybridoma Growth Media

OPI-HT Hybridoma Growth Media comprised: 600 ml of DMEM, High Glucose (4500 mg/L); 20 ml of 50× HT Supplement; 10 ml of 100× OPI Supplement; 10 ml of L. Glutamine; 200 ml (Standard=Characterized, Defined=Master) heat inactivated FBS; 200 ml of SP2/0 Conditioned Media; 10 ml of Penicillin/Streptomycin (100×); 1 ml of Amphotericin B (250 ug/ml stock); 5 ul of Beta-mercaptoethanol (98% pure in fume hood); Sterile Filter entire medium with 0.2 μm bottle-top filter to sterile IL bottle,

### Example 2—Characterization of Anti-Siglec-5 Antibodies

Monoclonal antibodies (mAbs) against Siglec-5 (generated in Example 1) were tested to determine if they recognize cell surface human Siglec-5 and whether they modulate a T cell antigen response.

### First ELISA

Tissue culture supernatants from the 1920 hybridomas were screened by ELISA against Siglec-5 (Cat #SIS-H5250 ACROBiosystems Inc.) and probed with an HRP-labeled Goat Anti-Mouse IgG secondary antibody. TMB (T5569-100 ml) was used in the reaction to visualize positives. H2SO4 or sulfuric acid was used to stop the reaction. Positive hybridoma clones >0.3 OD in this assay continued to the next level of development, and were subcloned.

### 24-Well Plate Culture of Positive Hybridomas

Positive cell lines were maintained in culture in 24-well culture plates after being transferred from the original 96 well fusion plates. The 24-well plates were grown for two weeks after which subcloning of the bybridoma occurred. 1 tube of cells was frozen from each well containing the fusion product that is moved forward into the 24 well format. During “nickel well” growth period two additional tubes of cells were frozen down. After subcloning, stabilization and validation of one of the two tubes after subcloning a master bank of tubes is made and from one of the master bank tubes 10 or more working bank tubes of hybridomas are frozen down. Generally, three rounds of subcloning was required to get an early stage hybridoma to a stable format in a production media.

### Hybridor a Cell Freezing

Hybridomas were frozen in Cell Freezing Media (20% DMSO) at an early stage or “Bulk” stage in order to limit the loss of particular clones. This procedure freezes hybridoma cell lines at this early stage “Bulk” and mid-stage ‘Mono’ hybridomas that are initially expanded in 24-well plates. No cell counting is necessary for this freezing technique.

### Subcloning of Hybridomas

The top hybridoma cell lines (clones) were subcloned to ensure monoclonality. Subcloning was performed by plating parental clones out again using our subcloning method. That method requires feeder cells at 50,000 per well in a 96 well plate. The feeder cells were a single cell suspension of a mouse splenocytes. Cells from a particular clone were drop wise placed in the 8 wells on the far left of the 96 well plate. In the 8 wells on the left the top well received the largest number of drops at 6 to 8 and the last well received a single drop of cells from the clone of interest. As the clones grow out during roughly a 6-10-day period the 96 well plate developed what is a bulk region on the left top and a monoclonal region on the lower far right of the plate. A minimum of 2 subclones were picked from the monoclonal region of the plate after screening by ELISA. Both top two clones were expanded in culture.

### Antigen (Siglec-5) Specific ELISA

For Ag-specific ELISA, 500 ng/well antigen (Siglec-5:Cat #SI5-HS250 ACROBiosystems Inc.) was coated to a 96-well plate. 25 ul of media from each of the 96 wells was incubated on the coated plates, and then washed with 0.05% Tween-20 in PBS (PBSST). A secondary goat anti-mouse “secondary antibody” conjugated to horseradish peroxidase (HRP) was used for detection of Siglec-5. After washings, the signal was detected by using TMB (tetramethylbenzidine), the reaction was stopped by the addition of IN H₂SO₄ stop solution and read at 450 nm on an ELISA plate reader from Molecular Devices (Sunnyvale, Calif.).

### Siglec-5 Present on Activated T-Cells-Flow Cytometry

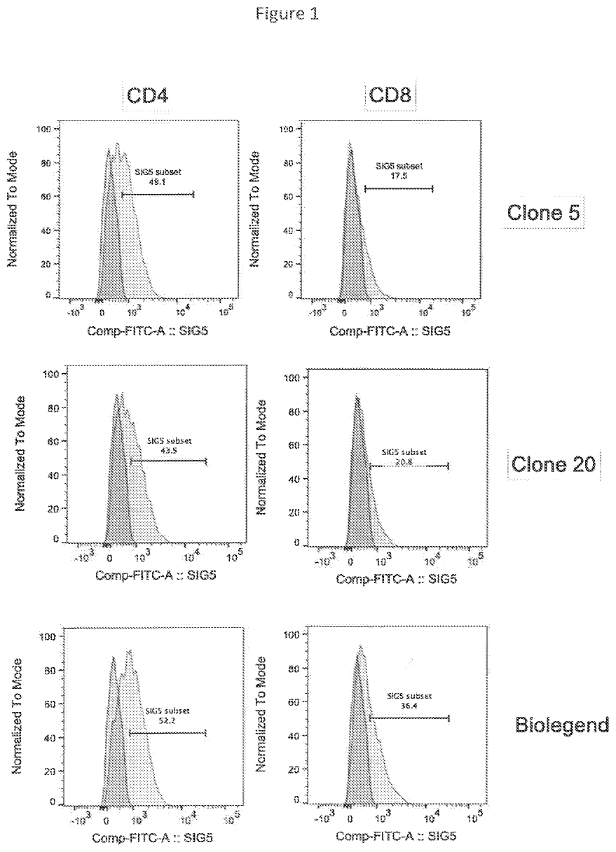

An aliquot of the cells was used to assess surface Siglec 5 staining (FIG. 1). For this assay, cells were stained with anti-Siglec 5 Abs (used at 1 μg/ml) for 30 minutes, washed, then re-stained with goat anti-mouse IgG-FITC Abs. Mouse IgG staining was used as a negative control. As a positive control, Biolegend monoclonal Ab was used (shown as BL). Using the Biolegend antibody as a control, it can be seen that the antibodies bind directly to both activated CD4 and CD& T cell populations. Only two representative clones (clones 5 and 20) are shown in FIG. 1. All other clones stained these cells at similar levels,

### Activated T-Cell Growth in the Presence of an ANTI-SIGLEC-5 Antibody

To prepare a population of activated T cells, human peripheral blood (from a healthy adult donor) derived mononucleated cells (PBMCs) were purified by Ficoll gradient. This preparation includes T cells, B cells, and myeloid cells (mostly monocytes). Cells (5×10⁶ cells per ml) were mixed with the polystyrene beads coated with anti-human CD3, and anti-human CD28 (50 μg/ml) in medium containing human IL-2 (10 ng/ml). After three days of culture, the cells were harvested.

The activated cells were then placed in secondary culture for a functional assay. Here, cells were restimulated with plate-coated anti-CD3 and anti-Siglec 5 mAbs (monoclonal antibodies) along with soluble anti-CD28 (no IL-2 was added). Culture supernatants from this assay were harvested after 1 and 4 days of culture for a multiplex cytokine assay. After 4 days of culture, cells were harvested and the total live cell count was determined for each mAb-treated sample. Here, anti-Siglec-5 and anti-PD1 mAbs from BioLegend, alone or in combination, were used as a reference point, and mouse IgG was used as a negative control for Siglec-5 mAbs,

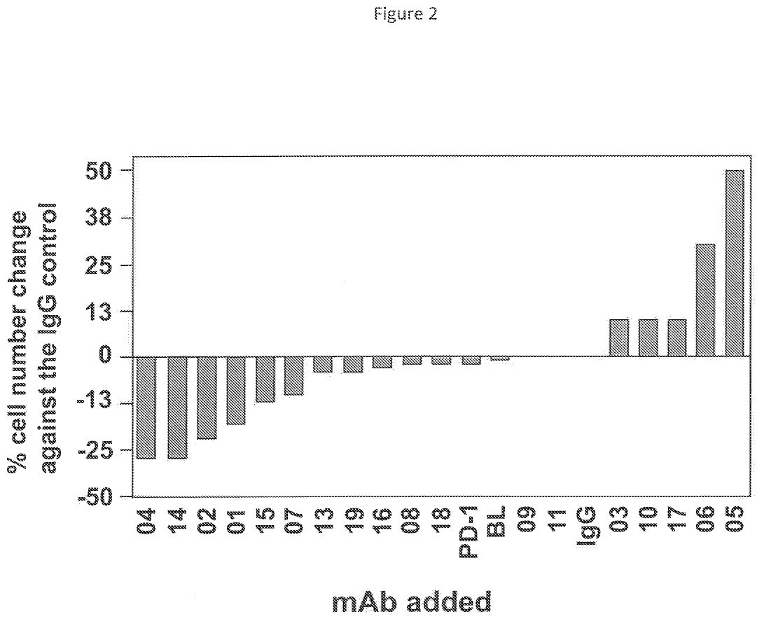

In contrast to the surface staining, cell growth analysis showed differences among these clones (FIG. 2). Antibodies that were positive for binding to human Siglec-5 were tested for their ability to bind activated T-cells and their ability to expand or grow out activated T-cell populations or to contract or decrease the growth of activated T-cell populations. The data in FIG. 2 show the percent difference in live cell count after 4 days of stimulation in the presence of anti-Siglec 5 Abs compared with mouse IgG control. Two clones showed more than a 10% increase in live cell count compared to the IgG control. Three other clones showed a mild increase. Five clones showed more than 10% reductions in cell count. In the first set of twenty monoclonal antibody clones, two clones (clone #5, #6) promoted activated T-cell growth by acting on the Siglec-5 present on both CD4 and CD8 positive activated T-cells. (FIG. 2).

The data show that all clones can recognize the surface-expressed human Siglec 5. The data also demonstrate that antibodies that bind Siglec-5 expressed on activated T cells can be identified as Siglec-5 agonists (useful for the treatment of e.g., autoimmune disorders) or as immune checkpoint inhibitors (useful for the treatment of e.g., cancer) by screening for their ability to modulate the T cell response.

### Example 3—Flow Cytometry-Siglec-5 Cytokine Profile and T-Cell Activation

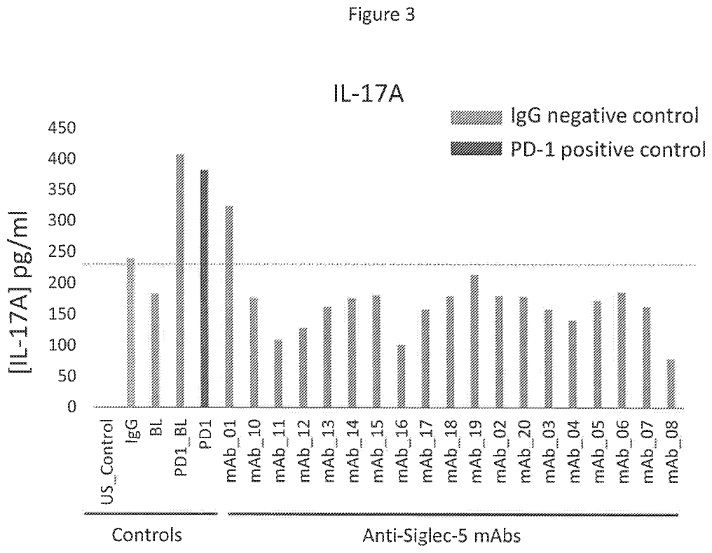

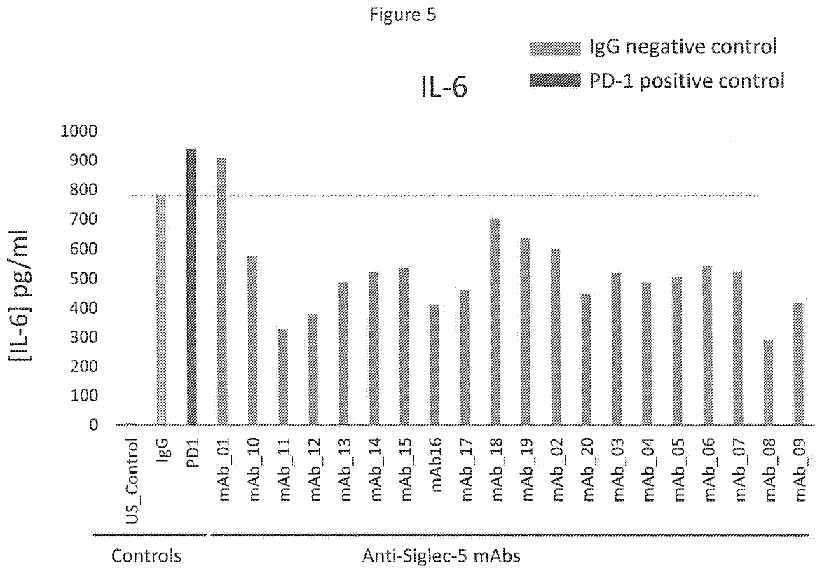

A population of activated human T cells from PBMC was prepared as described above. Following activation, the cells were placed in a secondary culture for a functional assay. Here, cells were restimulated with plate-coated anti-CD3 and anti-Siglec 5 mAbs (monoclonal antibodies) along with soluble anti-CD28 (no IL-2 was added). Culture supernates from this assay were harvested after 4 days of culture for multiplex cytokine assay. After 4 days of culture, cells were harvested and the cytokine profile of culture supernates was determined for each mAb-treated sample. IL-17-A, IL-2, and IL-6 cytokine profiles are shown in FIGS. 3-5. Here, anti-Siglec-5 and anti-PD1 mAbs from BioLegend, each as a single or as a mixture, were used as a reference point, and mouse IgG was used as a negative control for Siglec5 mAbs (monoclonal antibodies).

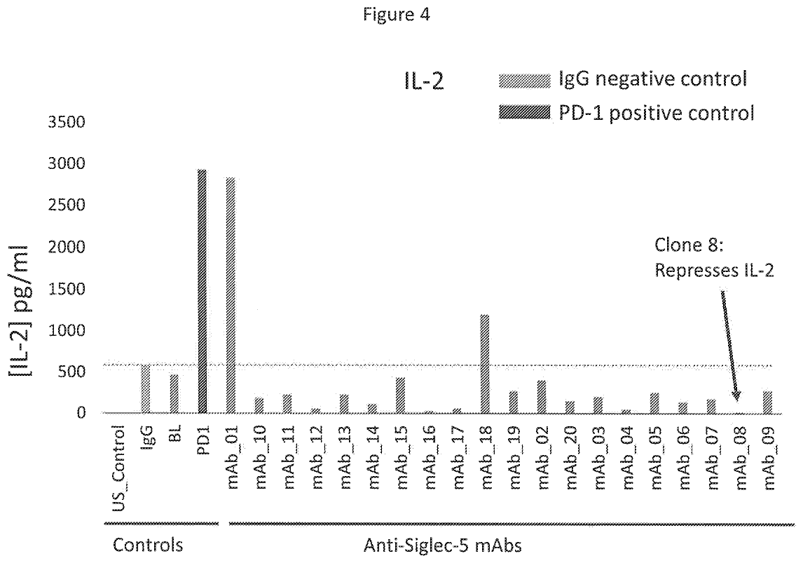

Assayed cytokines profiles during T-cell activation from culture supernates showed strong inhibition of IL-2, IL-17A and IL-6 production (FIGS. 3-5, clones 8, 11, and 16). In contrast, two clones (clone 1 and 18) substantially enhanced IL-2 and IL-17a production (FIGS. 3 and 4). Anti-PD1 mAb (monoclonal murine immune checkpoint inhibitor antibody) enhanced IL-2 and IL-17A and the effect was comparable between clone 1 and the anti-PD1 antibody. Both a decrease in T-cell activation and an increase in T-cell activation were observed depending on the mAb clone utilized in the assay, indicating that the anti-Siglec-5 antibodies are capable of modulating the T cell response.

### Example 4—Antigen-Specific Activation of Human 13831 TCR T-cells

The ability of the anti-Siglec-5 mAbs to act as immune checkpoint inhibitors by blocking the interaction between Siglec-5 on activated human 13831 TCR+ T cells (engineered human T cells transduced with a TCR (1383i) specific for melanoma antigen tyrosinase) and Siglec-5 ligand expressed on melanoma cells (MEL624 cells) was assessed.

Human 13831 TCR+ T cells were generated as described in Nishimura MI, et al., Cancer Res. 1999;59:6230-8. To stimulate the 13831 TCR+ T cells, the T cells were co-cultured with melanoma cell line MEL624 at a ratio of 10:1 (1383i T cells: MEL624). To this culture, purified anti-siglec-5 mABs (described in the previous examples) were added (5 μg/ml) from day 0. Culture supernatant was harvested 60 hours after the addition of the anti-Siglec-5 mAbs and analyzed by flow cytometry based cytokine assay (LEGENDplex by Biolegend),

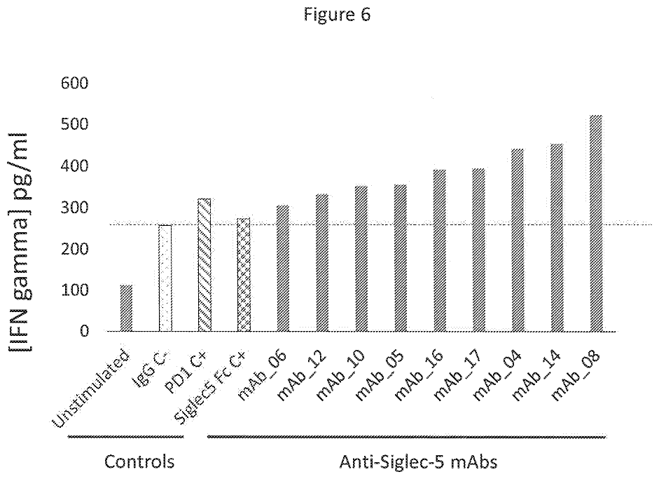

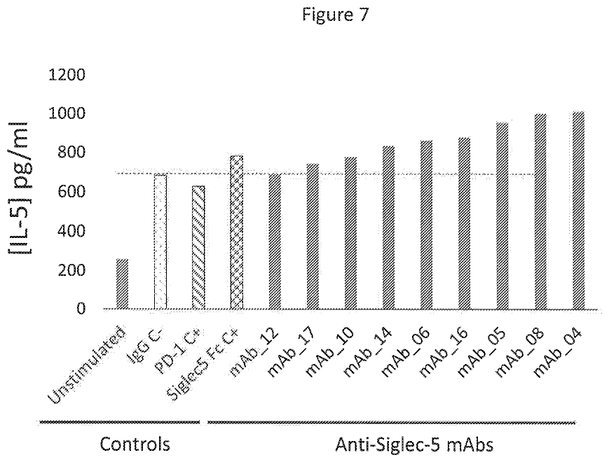

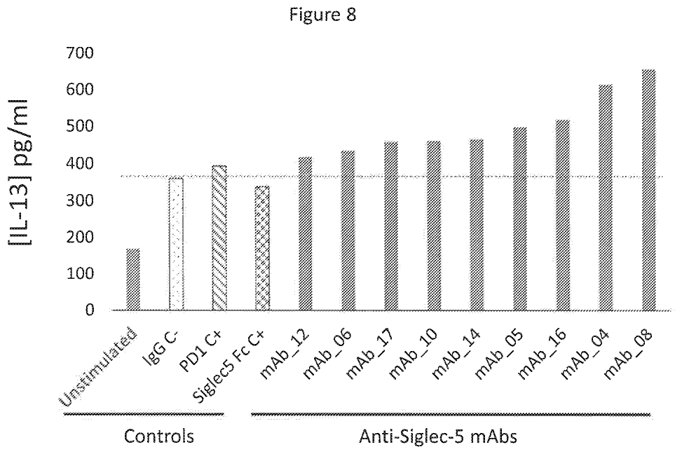

Analysis of the supernatant from melanoma-stimulated 1383i TCR T cells, showed an anti-Siglec-5 mAb-mediated increase in IFN gamma (FIG. 6), IL-5 (FIG. 7) and IL-13 (FIG. 8) expression by T cells treated with several of the mAb clones (clones 8 and 4 mediated a particularly strong response). Interestingly, the positive controls (Biolegend murine anti-PD-1 immune checkpoint inhibitor antibody (catalog #329902, which has a demonstrated ability to block PD-1 ligand binding) and soluble siglec-5, comprising the soluble portion of siglec-5 fused to an Fc fragment) only resulted in a slight T cell response in this assay (FIGS. 6-8, “PD1 C+” and “Siglec5 Fc C+”). Notably, in this assay, MEL624 does not produce any detectable level of cytokines tested, In stark contrast to the anti-Siglec-5 mAbs described herein, the Biolegend anti-Siglec-5 mAb did not have any significant observable effect on T cell stimulation (FIGS. 6-8).

The data demonstrate that several of the anti-Siglec-5 mAbs reverse T cell exhaustion resulting from engagement of Siglec-5 checkpoint protein by its ligand on the surface of melanoma cells. Interestingly, these anti-Siglec-5 mAbs generated a significantly stronger T cell response than the murine anti-human PD-1 immune checkpoint inhibitor (positive control) and soluble Siglec-5 (positive control). An even greater T cell response would be expected by the anti-Siglec-5 mAbs with longer stimulation times.

### Example 5—Use of Small Interfering Ribonucleic Acid Molecules in Silencing Expression of Siglec-5

An array of short interfering RNAs (shRNAs) about 15-25 nucleotides in length, and complementary to a region of human Siglec-5 mRNA, are designed. The shRNA bind to Siglec-5 mRNA and in the presence of the Dicer enzyme (a double stranded ribonuclease) form a “RISC complex” (RNA Interfering Silencing Complex) which cut the mRNA and render it unable to be translated, thus silencing the expression of the gene.

The shRNAs are introduced into one or more target cell(s) that express human Siglec-5 and the ability of the shRNAs to reduce (or knock-down) expression of Siglec-5 is assessed using an antibody-based detection assay (Enzyme-linked, immunosorbent assay [ELISA]) or the like. Preferably, the shRNAs are contained within a plasmid such that they are under the control of a promoter (e.g. bacteriophage T7 RNA Polymerase promoter) for continuous in vitro expression of the shRNAs. Alternatively, the shRNA is introduced into the target cell by electroporation, or viral-mediated viral transduction.

shRNAs are identified that result in decrease of Siglec-5 expression and thereby prevent the interaction of Siglec-5 with its ligand on one or more cancer cells. These shRNAs are assessed for anti-cancer activity in vitro in the one or more cancer cells and/or in vivo (in one or more suitable animal cancer models). Preferably the shRNAs are contained within a scaffold (e.g. a mir-30 backbone) that enhances knock-down of human Siglec-5.

Replicative plasmid(s) containing sequences of the shRNA, along with an expression promoter that is recognized by the cell RNA transcription machinery is used to generate siRNA within the target cell on a long-term basis; monitoring of Siglec-5 expression by the target cell/tissue is monitored by the same ELISA assay as described above. Alternatively, knock-down of Siglec-5 expression is accomplished using long double stranded RNA (long dsRNA) that follows the same mechanism as is described for shRNA.

### Example 6—Cooperative Antitumor Effect of PD-1 and Siglec-5 Blockade

Siglec-5 is co-expressed contemporaneously with PD-1 on activated T cells (data not shown). Co-administration of a compound that inhibits interaction of Siglec-5 on activated T cells with its cognate receptor on a cancer cell and a PD-1 inhibitor provides for synergistic antitumor effects by inhibiting multiple signal transduction pathways.

Synergistic effects on cancer are demonstrated by administering a combination of an anti-Siglec-5 antibody as described herein and an anti-PD-1 ICI antibody to a mouse model of cancer and comparing antitumoral effects observed with the combination to antitumoral effects observed in age-matched mice of the same cancer model administered the anti-Siglec-5 antibody or anti-PD-1 antibody alone. Synergistic (i.e. greater than additive) effects are demonstrated.

1. A method for inhibiting an interaction between an immune cell that expresses Siglec-5 and a cancer cell that expresses a Siglec-5 ligand comprising contacting the immune cell with a molecule that inhibits the interaction between Siglec-5 and the Siglec-5 ligand, wherein the molecule is a monoclonal antibody against Siglec-5.

2. The method of claim 1 wherein the method is performed in vitro.

3. The method of claim 1, wherein the method is performed in vivo.

4. The method of claim 1, wherein the immune cell that expresses Siglec-5 is an activated T-cell.

5. (canceled)

6. A method for the treatment of cancer comprising administering to a subject in need thereof a monoclonal antibody against human Siglec-5 or ligand-binding portion thereof that inhibits interaction between Siglec-5 expressed on an activated T cell and a Siglec-5 ligand expressed on the cancer cell(s).

7. (canceled)

8. The method of claim 6, wherein the monoclonal antibody against Siglec-5 or ligand-binding portion thereof increases production of at least one pro-inflammatory cytokine by at least 1.5-fold, at least 2-fold, at least 2.5-fold or at least 3-fold in a co-culture of (1) engineered human T cells transduced with a TCR (1383i) and (ii) MEL624 cells relative to the absence of the antibody or ligand-binding portion thereof, wherein the one or more pro-inflammatory cytokines comprises IFNγ.

9. (canceled)

10. The method of claim 6, wherein the antibody is a mouse antibody, a human antibody or is a humanized antibody.

11. (canceled)

12. The method according to claim 6, wherein the subject is identified as having a cancer that expresses a Siglec-5 ligand.

13. (canceled)

14. The method according to claim 12, wherein the cancer is selected from breast cancer, cervical cancer, ovarian cancer, colon cancer, colorectal cancer, rectal cancer, renal cancer, urothelial cancer, bladder cancer, prostate cancer, pancreatic cancer, intestinal cancer, anal cancer, head and neck cancer, pharynx cancer, gastro-esophageal cancer, esophageal cancer, nasopharyngeal cancer, thyroid cancer, Hodgkin's lymphoma, non-Hodgkin's lymphoma, T-cell lymphoma, T cell leukemia, B cell leukemia, a myeloid leukemia, a lymphoid leukemia, acute lymphocytic leukemia (ALL), acute myeloid leukemia (AML), acute promyelocytic leukemia (APML), chronic lymphocytic leukemia (CLL), chronic myeloid leukemia (CML), myeloid leukemia, Hairy cell leukemia, promyelocytic leukemia (PML), myelodysplastie syndrome (MDS), a lymphoproliferative disease, liver cancer, central nervous system cancer, skin cancer, and mesothelioma.

15. The method according to claim 14, wherein the cancer is selected from a hematological cancer, a leukemia, a melanoma, a liver cancer and a breast cancer.

16. The method according to claim 6, wherein the monoclonal antibody against Siglec-5 or ligand-binding portion thereof is co-administered to the subject with one or more additional immune checkpoint protein inhibitors.

17. The method according to claim 16, wherein the one or more additional immune checkpoint protein inhibitors comprise an inhibitor of an immune checkpoint protein selected from PD-1, PD-L1, CTLA-4, TIM-3, LAG-3, B7-H3 and B7-H4.

18. The method according to claim 17, wherein the one or more additional immune checkpoint protein inhibitors comprise an inhibitor of PD-1.

19. The method according to claim 17, wherein the monoclonal antibody against Siglec-5 or ligand-binding portion thereof and the one or more additional immune checkpoint protein inhibitors are administered concurrently.

20. The method according to any one of claim 16, wherein the subject is a human.

21. The method according to claim 20, wherein a tumor microenvironment of the subject comprises elevated expression of a Siglec-5 ligand and/or PD-1 relative to a control.

22-24. (canceled)

25. An isolated antibody or antigen-binding portion thereof, wherein the antibody or antigen-binding portion thereof binds to Siglec-5 and reduces binding of Siglec-5 to a Siglec-5 ligand.

26. The antibody or antigen-binding portion thereof according to claim 25, wherein the antibody or antigen-binding portion thereof specifically binds to human Siglec-5.

27. The antibody or antigen-binding portion thereof according to claim 25. wherein the antibody or antigen-binding portion thereof exhibits one or more of the following properties:
(a) binds to human Siglec-5 with a kD of [1×10−7 M] or less
(b) does not substantially reduce cell surface levels of Siglec-5 expressed on an immune cell (e.g. activated T cell) in vitro and/or in vivo
(c) increases T-cell proliferation in a Mixed Lymphocyte Reaction (MLR) assay
(d) increases IL-2 secretion in a MLR assay
(e) increases T-cell proliferation and/or increases IL-2 secretion in a T cell proliferation. assay
(f) binds to human Siglec-5 and cynomolgus monkey Siglec-5
(g) inhibits binding of Siglec-5 to a Siglec-5 ligand expressed on an immune cell
(h) inhibits tumor cell growth in vivo
(i) does not cross-react with human Siglec-14

28-29. (canceled)

30. A pharmaceutical composition, preferably for use in the treatment of cancer, comprising the antibody according to claim 25 and a pharmaceutically acceptable carrier.

31-34. (canceled)

35. A method of identifying a subject with cancer as a candidate for an anti-Siglec-5 treatment regimen, comprising assessing an expression level of Siglec-5 ligand on the cancer and/or in a tumor microenvironment, wherein a level of expression of Siglec-5 ligand on the cancer above a predetermined standard level identifies the subject as a candidate for the anti-Siglec-5 treatment regimen, optionally further comprising administering a molecule that inhibits interaction between Siglec-5 and the Siglec-5 ligand to a subject identified as a candidate for the anti-Siglec-5 treatment regimen.

36. (canceled)


SEQUENCE LISTING (1 sequence)

SEQ ID NO: 1 - protein, 551 aa, Homo sapiens
MLPLLLLPLLWGGSLQEKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQMKRQGAQVTTERTVQLNVSYAPQTITIFRNGIALEILQNTSYLPVLEGQALRLLCDAPSNPPAHLSWFQGSPALNATPISNTGILELRRVRSAEEGGFTCRAQHPLGFLQIFLNLSVYSLPQLLGPSCSWEAEGLHCRCSFRARPAPSLCWRLEEKPLEGNSSQGSFKVNSSSAGPWANSSLILHGGLSSDLKVSCKAWNIYGSQSGSVLLLQGRSNLGTGVVPAALGGAGVMALLCICLCLIFFLIVKARRKQAAGRPEKMDDEDPIMGTITSGSRKKPWPDSPGDQASPPGDAPPLEEQKELHYASLSFSEMKSREPKDQEAPSTTEYSEIKTSK